(19) United States
(12) Patent Application Publication
(10) Pub. No.: US 2026/0306316 A1
(43) Pub. Date: Oct. 8, 2026

(54) DEVICE FOR FIXING A LIVING BEING

(71) Applicant: KOHLBRAT & BUNZ Gesellschaft m.b.H., Radstadt (AT)

(72) Inventor: Michael GRAF, Zederhaus (AT)

(21) Appl. No.: 19/577,003

(22) Filed: Mar. 24, 2026

(30) Foreign Application Priority Data
Apr. 4, 2025 (AT) A 50241/2025

(51) Int. Cl. A61G 1/044; A61G 1/06

(52) U.S. Cl. CPC A61G 1/044; A61G 1/06; A61G 2203/10

(57) ABSTRACT

A device for fixing a living being, in particular a newborn, including a base element with a fastening means for fastening the device to a transport device, preferably a stretcher or a seat, and a flexible attachment element arranged on the base element. The attachment element can be fixed to the living being, the base element has a first evacuable or inflatable interior space, and the attachment element has a second evacuable interior space. The first interior space and the second interior space are designed separately from each other and/or can be evacuated separately from each other.

### BACKGROUND OF THE INVENTION

The present invention relates to a device for fixing a living being, in particular a newborn, comprising a base element with at least one fastening means for fastening the device to a transport device, preferably a stretcher or a seat, and at least one flexible attachment element arranged on the base element. The attachment element can be fixed to the living being, the base element has at least one first evacuable or inflatable interior space, and the at least one attachment element has at least one second evacuable interior space. Furthermore, the invention also relates to a method for fixing a living being with such a device.

Emergency transports of newborns or premature babies in ambulances represent a very intensive and difficult task for rescue organizations. This is particularly the case due to the high demands in the area of transport with regard to shocks, temperature maintenance, the possibility of ventilation and passing a 10G crash test.

A 10G crash test checks whether a living being, in this case a newborn, can survive an impact with ten times the force of gravity (10G) when placed in a suitable device. If this is the case, the device meets the requirements of a 10G crash test. In particular, this latter safety-related consideration is becoming increasingly important for devices of this type. Accordingly, there is a need for a restraint system to secure newborns in vehicles that meets both medical requirements and the requirements regarding the aforementioned safety aspects.

The ideal embodiment of such a device in this regard is a special incubator for newborns, which is suitable for use in vehicles. In practice, specially equipped rescue vehicles (children's ambulances) are provided for this purpose. However, these are not available in sufficient numbers and therefore not across the board for rescue organizations. Furthermore, such vehicles can only be used for transporting premature newborns or infants and are mostly used for transfers between clinics. Mobile incubators, such as those used in helicopters, are only used for transport between clinics.

For regular and scheduled emergency medical services, other systems are currently used in the form of differently formed stretchers for emergency transport to transport (adult) patients to the emergency room in the ambulance.

Systems are also known for securing newborns to existing stretchers in vehicles using strap systems and padding. These systems are mostly simple belt solutions, designed to provide at least some restraint during transport. There are also known restraint systems that can be combined with special vacuum mattresses.

Even with a special vacuum mattress, specially adapted straps and buckles, or using vacuum mattresses with built-in fixation options, the fixation of newborns in such systems does not meet medical and safety requirements.

The main problem is that newborns are too small in terms of body size to be securely restrained with strap or buckle systems. It is almost impossible to secure the straps without a buckle or anything else touching the patient, which can lead to pressure sores or, in the worst case, injuries.

### SUMMARY OF THE INVENTION

The object of the invention is therefore to specify a generic device which can be used in conventional (rescue) vehicles and which meets the new medical and safety requirements. Furthermore, a method for fixing a living being with such a device is also to be specified.

According to the invention, in a device as described above, the at least one first interior space and the at least one second interior space are designed separately from each other and/or can be evacuated separately from each other. The base element and the attachment element can therefore be evacuated and shaped separately. The base element can therefore serve as a basis for fixing the living being as well as for securing the device in a vehicle (to a transport device of the vehicle).

For the latter, the device has at least one fastening means, for example straps and buckles, on the base element to fix the base element to the existing stretcher or to securely connect it to other fixed objects (such as car seats) or mobile stretchers in hospitals. This allows the device to be fastened to a vehicle in such a way that it meets the safety requirements, especially with regard to a 10G crash test.

Another advantage is that this solution can be fastened not only in the middle of a stretcher, but can be flexibly mounted across the entire region of the stretcher. This allows the device to be positioned within the region of an accompanying person in a (rescue) vehicle, enabling the accompanying person in the “rescue seat” in the vehicle to work on the newborn without having to release their safety belt.

Furthermore, after the first interior space has been evacuated, the base element can be used as a kind of stable board and act like a spine board, thus immobilizing the living being to a certain degree.

The attachment element can be molded to the newborn before evacuation and represents a negative impression of the newborn body. After the attachment element has been evacuated, it fixes the newborn in the longitudinal and transverse axes. In combination with the base element, this results in the newborn being enclosed on at least three sides – which prevents the newborn from moving. This allows for sufficient medical fixation in the head and neck region, or lateral stability, which sufficiently stabilizes or immobilizes even newborns with their underdeveloped spine and neck muscles. Movement of the newborn is therefore prevented as much as possible.

However, the design of the attachment element also allows for an open and accessible region towards the head and chest of the newborn. This allows the newborn to be ventilated or resuscitated if necessary while it is in the device. The separate design and evacuation capacity of the first and second interior spaces thus allow, on the one hand, better “hardening” of the base element or the attachment element during evacuation, and on the other hand, better molding of the aforementioned elements to the body of a living being.

In an evacuated state, such a device initially prevents undercooling or the removal of heat on its own due to the vacuum. The device forms a specially shaped vacuum mattress. The interior spaces of the device are therefore evacuated in the manner of a vacuum mattress.

The above statements apply analogously in the case where the base element is inflated instead of evacuated. The base element can be inflated to such an extent that it becomes rigid, as is the case, for example, with inflatable stand-up paddle boards.

A method according to the invention comprises the following steps:

placing the living being on the base element,

evacuating or inflating the first interior space, wherein the base element is hardened in such a way that a spine of the living being is at least partially immobilized,

molding the attachment element to fit the living being, and

evacuating the second interior space so that the living being is supported and/or stabilized in the device.

These steps do not have to be carried out in the specified order, but can also be performed in a different order or simultaneously or partially overlapping.

Advantageously, the base element may be provided with a support section on which the living being can be placed. The attachment element can be detachably or permanently connected to the base element. This allows for better stabilization of a living being via the device.

The at least one attachment element can be formed in a U-shape. The U-shaped design of the attachment element allows it to be easily and precisely molded to fit the body of a living being. Especially when the living being is a newborn, the attachment element can completely enclose the newborn body laterally as well as in the region of the head.

Preferably, the device may have at least one first valve for evacuating or inflating the base element and at least one second valve for evacuating the at least one attachment element. This provides a simple way to evacuate the first interior space and the second interior space separately from each other. However, it is also conceivable that the first and second interior spaces have a common valve, with the valve being designed to evacuate the first and second interior spaces separately from each other.

In a state in which a living being is supported and/or stabilized in the device, at least a head and a chest region of the living being stabilized and/or supported in the device can be accessible. This is particularly advantageous because it allows the living being to be resuscitated and/or ventilated, for example, while it is securely fixed in the device.

Preferably, granules are arranged in the first interior space and in the second interior space. The use of granules can improve the fitting molding of the attachment element to the living being and the hardening of the base element during evacuation.

According to a further embodiment, at least one damping element, preferably comprising a spacer fabric and/or a foam, is arranged in the first interior space and/or in the second interior space. This eliminates vibrations in different frequency ranges, which can be caused, for example, by transport in a vehicle. This results in a more stable and calmer support for the living being. Such a damping element can also serve as padding for the living being in the device.

Advantageously, at least one fastening device can comprise at least one strap and/or at least one buckle. This represents a simple option for implementing a fastening means. Furthermore, such a fastening means is universally applicable, for example to differently designed stretchers and/or seats. However, other designs of a fastening means are also conceivable.

Handles and/or retaining straps separate from the at least one fastening means can be arranged on the device, preferably on the base element. This makes it easier, for example for rescue workers, to transport and handle a device.

According to another exemplary embodiment, the base element can be rectangular. A rectangular base element is easy to manufacture, easy to handle, and compatible with a variety of stretchers and/or seats. Of course, other forms for the base element are also conceivable.

The at least one attachment element can comprise at least one securing device, preferably a strap, to additionally fix the living being in the attachment element. The attachment element can be closed or fixed by means of at least one securing device. This allows for additional fixation of the living being within the device. This is particularly advantageous in the event of incorrect or faulty fitting molding of the attachment section to the living being. However, with such a securing device, access to the living being from above may still be possible.

Particularly preferably, the device may include at least one temperature control device for temperature-controlling a living being, in particular for cooling and/or heating. Such a temperature control device makes it possible to regulate the temperature of a living being within the device. The living being can, for example, be warmed to prevent hypothermia. It is also possible, for example, to cool the living being in order to avoid damage, for example to the brain. At least one temperature control device can be arranged in the first interior space and/or in the second interior space. Such direct integration into the device eliminates the need for a separate temperature control device, making the device easier to handle.

Particularly preferably, the temperature control device may comprise, for temperature-controlling a living being, in particular a human being:

a shell with a first side for the support of a living being, and

at least one temperature control element which is arranged within the shell, wherein a heat flow can be generated by the at least one temperature control element.

The temperature control device includes at least one heat transfer element arranged within the shell, by which the heat flow generated by the at least one temperature control element can be transferred to the first side of the shell in order to warm and/or cool a living being located on the shell. This makes it possible to provide a heat flow via the temperature control element, which has a warming and/or cooling effect on the first side of the shell. Accordingly, a living being located on the shell can be warmed and/or cooled.

A heat transfer element has the advantage that a large-area temperature control element is not required. This allows for the use of small temperature control elements, which are cost-effective and less susceptible to damage. An additional stabilizing layer can also be omitted.

Regarding a cooling temperature control element, a heat transfer element allows for a meaningful implementation of a temperature control device with sufficient cooling capacity in the first place. Flat temperature control elements that can produce a cooling effect and are suitable for use in temperature control devices of this type are either unknown or too expensive.

With regard to a method, it may be provided that the device is fastened in a further step to a transport device, preferably a stretcher or a seat, via the at least one fastening means.

### BRIEF DESCRIPTION OF THE DRAWINGS

Further details and advantages of the invention will be explained in more detail hereinafter with reference to the drawings, in which:

FIG. 1A is a schematic top view of a device,

FIG. 1B is a schematic bottom view of a device,

FIG. 2 is a schematic top view of the device with a living being fixed therein,

FIG. 3 is a schematic top view of the device on a transport means,

FIG. 4A is a schematic sectional view of a first exemplary embodiment of the device,

FIG. 4B is a schematic sectional view of a second exemplary embodiment of the device, and

FIG. 4C is a schematic sectional view of a third exemplary embodiment of the device.

### DETAILED DESCRIPTION OF THE INVENTION

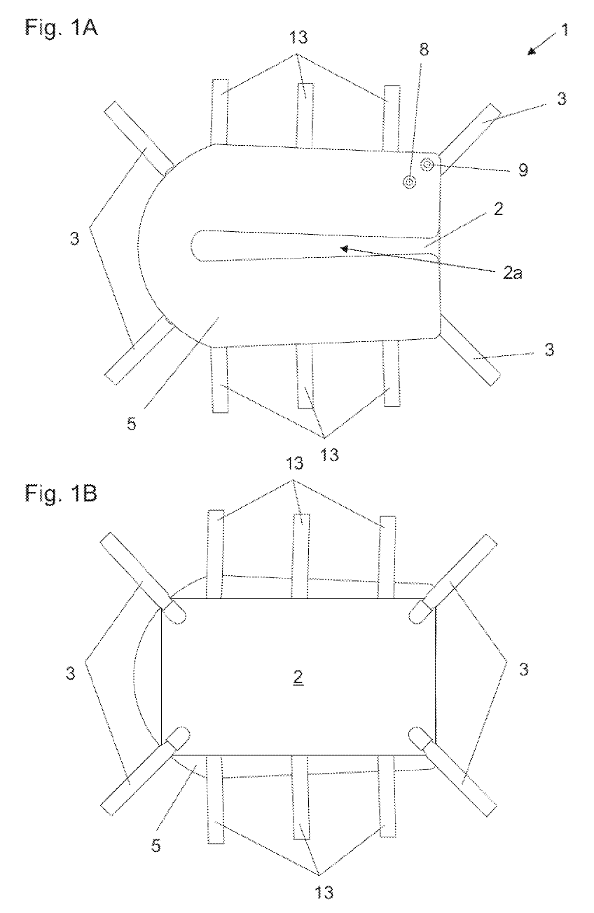

FIG. 1A shows a schematic top view of a device 1 and FIG. 1B shows a schematic bottom view of the device 1. It can be seen that the device 1 has a substantially rectangular base element 2, on which fastening means 3 are arranged. In the present exemplary embodiment, the fastening means 3 are designed as straps. However, other types of fastening means 3 are also conceivable. An attachment element 5 connected to the base element 2 is arranged on the base element 2. The attachment element 5 is approximately U-shaped.

In this exemplary embodiment, both the base element 2 and the attachment element 3 are designed to be elastic. The attachment element 3 can be molded to fit the body of a living being. A support section 2a is formed on the base element 2, on which support section a living being can come to rest. The support section 2a may also extend partially below attachment section 5.

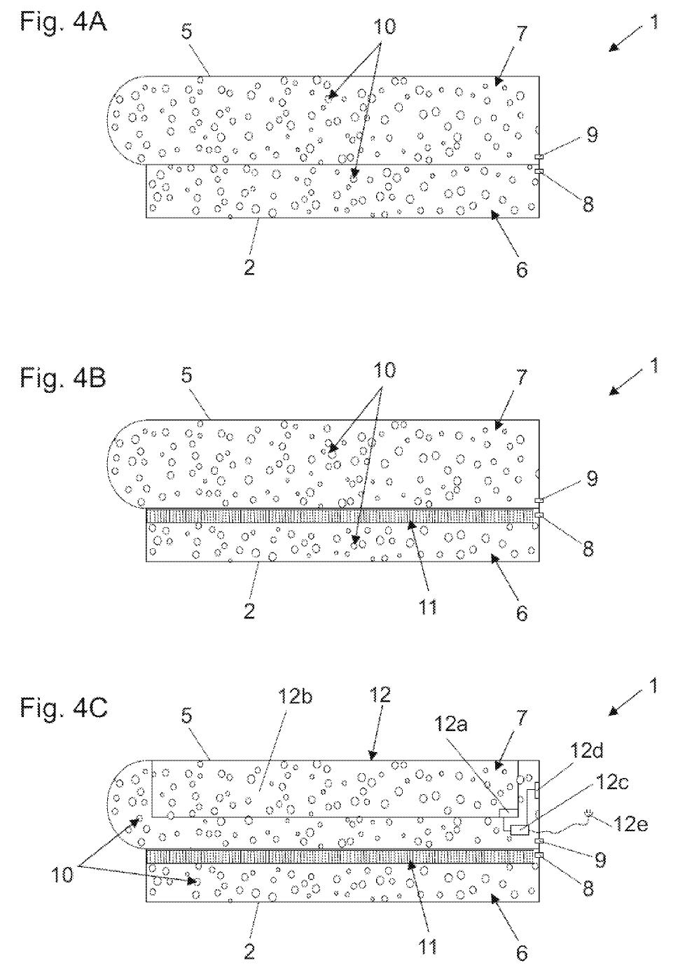

In addition, a first valve 8 for evacuating or inflating the base element 2 and a second valve 9 for evacuating the attachment element 5 are visible. In the present embodiment, the first valve 8 can be guided through the attachment element 5 to the base element 2. In principle, the first valve 8 and the second valve 9 can be placed at any location on the device 1. An alternative placement can be seen, for example, in FIGS. 4A-4C.

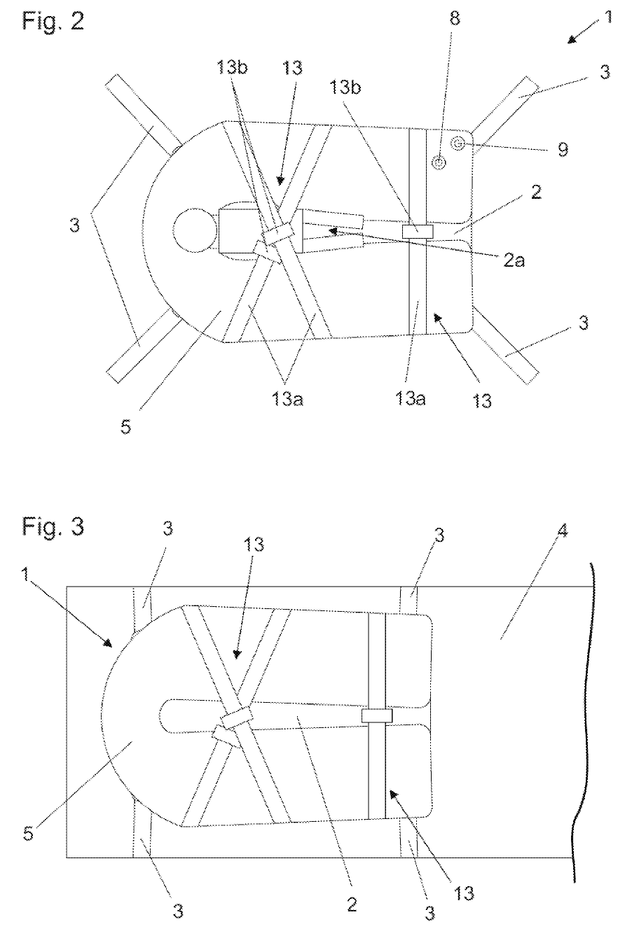

Multiple securing means 13, each with a strap 13a and a buckle 13b, can also be seen on the attachment element 5. These securing means 13 can hold together the two legs of the U-shaped attachment section 5. In other words, the attachment element 5 can be closed or fixed. This allows for additional fixation of the living being within the device. This is clearly shown in FIG. 2, which shows a schematic top view of the device 1 with a living being fixed therein. The living being in FIG. 2 is a newborn, which can be recognized by its small size.

The attachment section 5 has been molded around the living being and evacuated. The base element 2 has also been evacuated. The base element 2 is therefore hardened and forms a kind of “hard board”. The base element 32 thus functions as a spine board for the living being and, in particular, immobilizes the spine of the living being. It is also conceivable that the base element 2 is inflated to harden it.

The living being is securely fixed in the device 5 via the molded-on attachment section. In addition, the securing means 13 are also in place, which allow for additional fixing of the living being. Full access to the living being in the head and chest region from above is provided in order to be able to ventilate and/or resuscitate the living being if necessary.

FIG. 3 shows a schematic top view of the device 1 on a transport means 4. The transport means 4 is designed here as a stretcher. The device 1 is connected to the transport device 4 via the fastening means 3. This connection is designed in such a way that all safety requirements are met. In particular, it can also pass a 10G crash test. The transport device 4 can also be, for example, a (vehicle) seat, a hospital bed or the like.

FIG. 4A shows a schematic sectional view of a first exemplary embodiment of the device 1. It can be seen that the base element has a first interior space 6 and the attachment element 5 has a second interior space 7. The first interior space 6 and the second interior space 7 are designed separately from each other and can also be evacuated separately from each other. For the latter, a first valve 8 connected to the first interior space 6 and a second valve 9 connected to the second interior space 7 are provided. Granules 10 are arranged in both the first interior space 6 and the second interior space 7. In the non-evacuated state, the attachment element 5 can be molded to fit the living being, wherein the granules 10 in the second interior space 7 deform accordingly. After evacuating the second interior space 7, the attachment element 5 is hardened in its molded form due to the granules 10, thus fixing the living being. In the base element 2, a hardening of the base element 2 can be achieved via the granules.

FIG. 4B shows a schematic sectional view of a second exemplary embodiment of the device 1. This differs from the first exemplary embodiment only in that a damping element 11 in the form of a spacer fabric is arranged in the first interior space 6. Vibrations in the device can be reduced and/or eliminated via the damping element 11. The damping element 11 can also be provided only in the second interior space 7 or in both interior spaces 6, 7.

FIG. 4C shows a schematic sectional view of a third exemplary embodiment of the device 1. This differs from the second exemplary embodiment in that a temperature control device 12 is provided in the second interior space 7. The temperature control device 12 comprises a temperature control element 12a for generating a heat flow. In physical terms, heat flow is understood as the amount of heat that is transferred from a place of high temperature to a place of low temperature. Depending on the direction of the heat flow, the heat flow generated by the temperature control element can therefore result in either warming or cooling. Here, a shell of the temperature control device 12 is formed by a wall of the attachment element 5 or of the second interior space 7.

The temperature control device 12 further comprises one heat transfer element 12b, by means of which the heat flow generated by the at least one temperature control element 12a can be transferred to the first side of the shell in order to warm and/or cool a living being located on the shell. Thus, the temperature of a living being fixed in the device can be controlled using the temperature control device 12. The temperature control element 12a can, for example, comprise a Peltier element. The heat transfer element 12b can comprise a thermally conductive film.

The temperature control device 12 also comprises a regulating device 12c for regulating and/or controlling the temperature control element 12a. In addition, an actuating element 12d is also provided for operating the temperature control device 12. The temperature control device 12 can be supplied with energy via an interface 12e in the form of a plug.

It is also conceivable that a temperature sensor is provided, wherein the temperature control device 12 can be regulated depending on the measured values of the temperature sensor. The temperature control device 12 can also be provided in only the first interior space 6 or in both interior spaces 6, 7.

### LIST OF REFERENCE NUMERALS

1 device
2 base element
2a support section
3 fastening means
4 transport device
5 attachment element
6 first interior space
7 second interior space
8 first valve
9 second valve
10 granules
11 damping element
12 temperature control device
12a temperature control element
12b heat transfer element
12c regulating device
12d actuating element
12e interface
13 securing means
13a strap
13b buckle

1. A device for fixing a living being, in particular a newborn, comprising:
a base element with at least one fastening means for fastening the device to a transport device, preferably a stretcher or a seat, and
at least one flexible attachment element arranged on the base element, wherein the attachment element can be fixed to the living being,
wherein the base element has at least one first evacuable or inflatable interior space and the at least one attachment element has at least one second evacuable interior space, wherein the at least one first interior space and the at least one second interior space are designed separately from each other and/or can be evacuated separately from each other.

2. The device according to claim 1, wherein the base element has a support section on which the living being can be placed for support.

3. The device according to claim 1, wherein the at least one attachment element is U-shaped.

4. The device according to claim 1, wherein the device has at least one first valve for evacuating or inflating the base element and at least one second valve for evacuating the at least one attachment element.

5. The device according to claim 1, wherein in a state in which a living being is fixed in the device at least a head and a chest region of the living being fixed in the device is accessible.

6. The device according to claim 1, wherein granules are arranged in the first interior space and in the second interior space respectively.

7. The device according to claim 1, wherein at least one damping element, preferably comprising a spacer fabric and/or a foam, is arranged in the first interior space and/or in the second interior space.

8. The device according to claim 1, wherein the at least one fastening means comprises at least one strap and/or at least one buckle.

9. The device according to claim 1, wherein handles and/or retaining straps separate from the at least one fastening means are arranged on the device, preferably on the base element.

10. The device according to claim 1, wherein the base element is rectangular.

11. The device according to claim 1, wherein the device comprises at least one temperature control device for temperature-controlling a living being, in particular for cooling and/or warming.

12. The device according to claim 11, wherein the at least one temperature control device is arranged in the first interior space and/or in the second interior space.

13. The device according to claim 11, wherein the temperature control device for temperature-controlling a living being, in particular a human being, comprises:
a shell with a first side for the support of a living being, and
at least one temperature control element which is arranged within the shell, wherein a heat flow can be generated by means of the at least one temperature control element,
wherein the temperature control device comprises at least one heat transfer element arranged within the shell, by means of which the heat flow generated by the at least one temperature control element can be transferred to the first side of the shell in order to warm and/or cool a living being located on the shell.

14. A method for fixing a living being, in particular a newborn, with a device according to claim 1, comprising:
placing the living being on the base element,
evacuating or inflating the first interior space, wherein the base element is hardened in such a way that a spine of the living being is at least partially immobilized,
molding the attachment element to fit the living being, and
evacuating the second interior space so that the living being is supported and/or stabilized in the device.

15. The method according to claim 14, wherein the device is fastened in a further step to a transport device, preferably a stretcher or a seat, via the at least one fastening means.
